(19) United States
(12) Patent Application Publication
(10) Pub. No.: US 2026/0141514 A1
(43) Pub. Date: May 21, 2026

(54) EXPLAINABILITY HEAT MAP FOR AUTONOMOUS DIAGNOSTICS BASED ON HIGH RESOLUTION MEDICAL IMAGE PROCESSING USING VISION TRANSFORMER

(71) Applicant: Digital Diagnostics Inc., Coralville, IA (US)

(72) Inventor: Evan Cary Harvey, Sparks, NV (US)

(21) Appl. No.: 18/955,627

(22) Filed: Nov. 21, 2024

(51) Int. Cl. G06T 7/00; G16H 30/40; G16H 50/20

(52) U.S. Cl. CPC G06T 7/0012; G16H 30/40; G16H 50/20

(57) ABSTRACT

A disease diagnosis tool validates a diagnosis of a patient. The disease diagnosis tool receives a high-resolution image of a body part. The disease diagnosis tool divides the high-resolution image into a plurality of tiles and inputs a representation of each tile into an encoder portion of a model configured to perform a disease diagnosis based on the representations. The encoder has an attention mechanism. The disease diagnosis tool obtains a plurality of tokens representative of an attention of the encoder based on the attention mechanism. Each token is associated with a position of a tile of the plurality of tiles. The disease diagnosis tool generates a heat map corresponding to the image of the body part. The heat map comprises a two-dimensional image having pixels corresponding to tiles each with an amplitude based on a level of attention represented in the plurality of tokens corresponding to the tiles.

### BACKGROUND

Machine learning approaches for analyzing images involve inputting images into models that identify the content of the input images. One such model is a vision transformer (ViT). A vision transformer (ViT) is an image classification model that employs a transformer-based architecture. Much like how a transformer breaks text into a series of tokens and draws meaning from comparisons between the tokens, a vision transformer breaks an image into smaller image segments and draws meaning from comparisons between the image segments. As a vision transformer's attention layers compute interactions between every image segment, the cost of deploying a vision transformer increases dramatically with the size of the image being processed. Typically, systems solve this cost issue by downscaling images before applying a vision transformer.

Machine learning approaches to autonomously diagnosing disease use images of body parts to determine whether features within the image are indicative of disease. Due to the practical need to downscale images, vision transformers have not been used in autonomous medical diagnosis. That is, downsizing these high-resolution images results in loss of image information that is critical for making accurate diagnoses, in that even minor blood vessels taking up small numbers of pixels that are crucial for diagnosis can be lost during downscaling. The results of vision transformers can also suffer from lack of explainability, thereby precluding use for autonomous diagnosis because accuracy cannot be verified.

### SUMMARY

Systems and methods are disclosed herein that use a machine learning approach employing a vision transformer to autonomously determine a diagnosis of a disease based on an image of a body part. A diagnosis tool uses a vision transformer to determine a diagnosis of a disease from a high-resolution image of a body part. Rather than downsizing the high-resolution image, the disease diagnosis tool divides the high-resolution image into tiles and computes embeddings for the tiles. By computing tile embeddings, the disease diagnosis tool reduces the dimensionality of the tiles without losing valuable information. The disease diagnosis tool may then apply the vision transformer to the computed embeddings. The disease diagnosis tool generates a heatmap representing the attention the vision transformer placed on different areas of the high-resolution image. In generating the heatmap, the disease diagnosis tool allows medical experts to confirm the relevance of each area of the high-resolution image to the diagnosis output by the vision transformer and ultimately determine whether the diagnosis was accurate.

In an embodiment, a disease diagnosis tool receives a high-resolution image of a body part. The disease diagnosis tool divides the high-resolution image into a plurality of tiles. For each tile, the disease diagnosis tool generates an embedding having a position encoding corresponding to the tile's position in the high-resolution image. The disease diagnosis tool inputs the embeddings for the tiles into a linear projection model whose output feeds a transformer and receives, as output from a model comprising the transformer, a diagnosis of a disease of the body part.

In an embodiment, a disease diagnosis tool validates a diagnosis of a patient. The disease diagnosis tool receives a high-resolution image of a body part. The disease diagnosis tool divides the high-resolution image into a plurality of tiles. The disease diagnosis tool inputs a representation of each tile into an encoder portion of a model configured to perform a disease diagnosis based on the representations. The encoder has an attention mechanism. The disease diagnosis tool obtains a plurality of tokens representative of an attention of the encoder based on the attention mechanism. Each token is associated with a position of a tile of the plurality of tiles. The disease diagnosis tool generates for display a heat map corresponding to the image of the body part. The heat map comprises a two-dimensional image having pixels corresponding to tiles each with an amplitude based on a level of attention represented in the plurality of tokens corresponding to the tiles.

### BRIEF DESCRIPTION OF THE DRAWINGS

FIG. 1 illustrates one embodiment of a system environment for implementing a disease diagnosis tool.

FIG. 2 illustrates one embodiment of exemplary modules and databases used by the disease diagnosis tool in using machine learning models to output a diagnosis of a disease of a body part.

FIG. 3A illustrates an example tiled image for an image of a body part.

FIG. 3B illustrates example tile embeddings for an image of a body part.

FIG. 4 illustrates an example vision transformer.

FIG. 5 illustrates example pipelines for determining a diagnosis from an image of a body part.

FIG. 6 illustrates example heatmaps for images of eyes.

FIG. 7 is a flowchart of an exemplary process for using a vision transformer to produce a diagnosis from high-resolution images, in accordance with an embodiment.

FIG. 8 is a flowchart of an exemplary process for generating a heatmap representative of attention placed on pixels of an image by a vision transformer, in accordance with an embodiment.

The figures depict various embodiments of the present invention for purposes of illustration only. One skilled in the art will readily recognize from the following discussion that alternative embodiments of the structures and methods illustrated herein may be employed without departing from the principles of the invention described herein.

### DETAILED DESCRIPTION

### (a) Overview

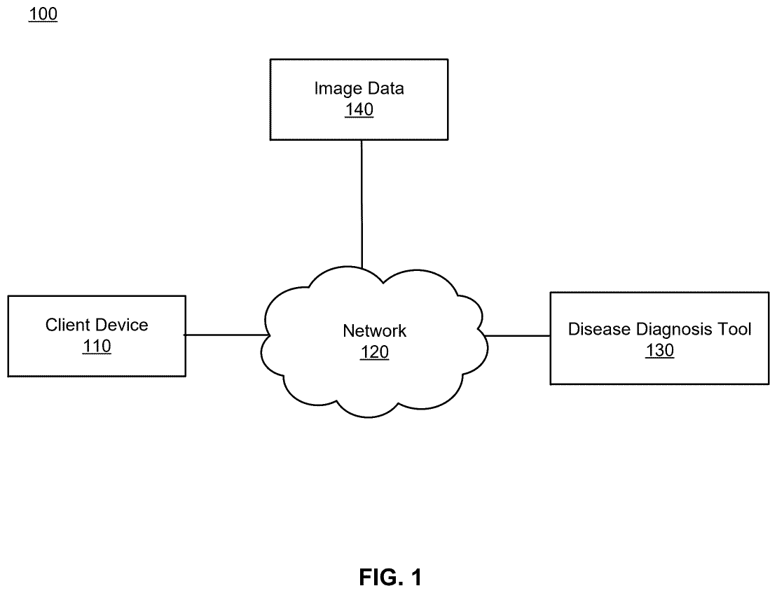

FIG. 1 illustrates one embodiment of a system environment for implementing a disease diagnosis tool. As depicted in FIG. 1, environment 100 includes client device 110, network 120, the disease diagnosis tool 130, and image data 140. The elements of environment 100 are merely exemplary; fewer or more elements may be incorporated into environment 100 to achieve the functionality disclosed herein.

Client device 110 is a device in which inputs of image data may be provided, and where diagnoses may be output, the diagnoses determined by the disease diagnosis tool 130 based on the image data. Client device 110 may run an application installed thereon, or may have a browser installed thereon through which an application is accessed, the application performing some or all functionality of the disease diagnosis tool 130 and/or communicating information to and from the disease diagnosis tool 130. The application may include a user interface through which a user can input image data into client device 110. The user interface may be graphical, where the user can input image data manually (e.g., through a keyboard or touch screen). For example, the user may upload an image taken from an external image sensor (e.g., a camera). The user interface may additionally or alternatively be operably coupled to an image sensor, where image data is captured and transmitted by the application to the disease diagnosis tool 130. For example, the user interface may allow the user to capture an image using a camera of the client device 110. The user interface may be used to access existing image data stored in image data 140, which may then be communicated to the disease diagnosis tool 130 for processing.

Client device 110 may be any device capable of transmitting data communications over network 120. In an embodiment, client device 110 is a consumer electronics device, such as a laptop, smartphone, personal computer, tablet, personal computer, and so on. In an embodiment, client device 110 may be any device that is, or incorporates, a sensor that senses patient data (e.g., motion data, blood saturation data, breathing data, or any other biometric data).

Network 120 may be any data network capable of transmitting data communications between client device 110 and the disease diagnosis tool 130. Network 120 may be, for example, the Internet, a local area network, a wide area network, or any other network.

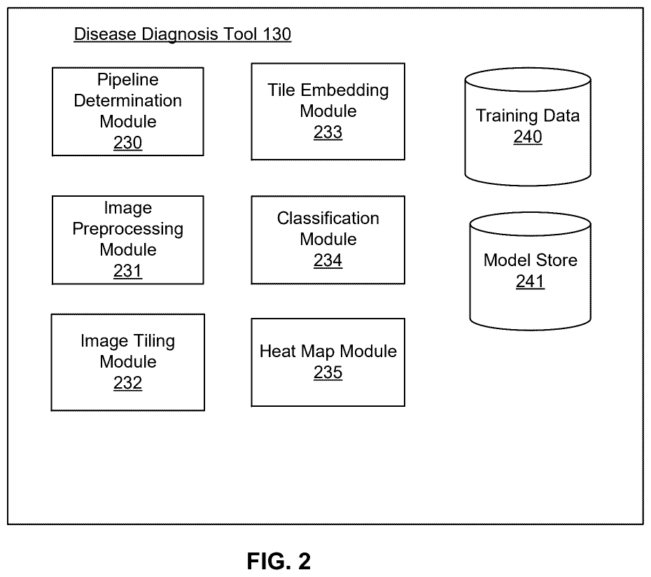

The disease diagnosis tool 130 receives images of body parts from client device 110 and outputs autonomous diagnoses of diseases of the body parts. Further details of the operation of the disease diagnosis tool 130 are discussed below with reference to FIG. 2. Operations of the disease diagnosis tool 130 may be instantiated in whole or in part on client device 110 (e.g., through an application installed on client device 110 or accessed by client device 110 through a browser).

Image data 140 is a database that stores images of body parts of one or more individuals for use in diagnosing diseases. Images may be of an external body parts, such as high-resolution images of eyes or skin, or internal body parts, such as images of retinas or livers. The image may be any type of image, for example including a grayscale image, a red-green-blue image, an infrared image, an x-ray image, an optical coherence tomography image, a sonogram image, or any other type of image. The images may be captured by any type of image sensor or imaging system. For example, image data 140 of retinas may be captured by optical coherence tomography (OCT) or scanning laser ophthalmoscopy (SLO) systems. Image data 140 may include images taken by the client device 110, for example images captured with a camera of the client device 110. Image data 140 may be co-located at client device 110 and/or at the disease diagnosis tool 130.

FIG. 2 illustrates one embodiment of exemplary modules and databases used by the disease diagnosis tool in using a machine learning approach to autonomously determine a diagnosis of a disease of a body part. As depicted in FIG. 2, the disease diagnosis tool 130 includes pipeline determination module 230, image tiling module 232, tile embedding module 233, classification module 234, heat map module 235, an image preprocessing module 231, training data 240, and model store 241. The modules and databases depicted in FIG. 2 are merely exemplary; the disease diagnosis tool 130 may include more or fewer modules and/or databases and still achieve the functionality described herein. Moreover, the modules and/or databases of the disease diagnosis tool 130 may be instantiated in whole, or in part, on client device 110 and/or one or more servers.

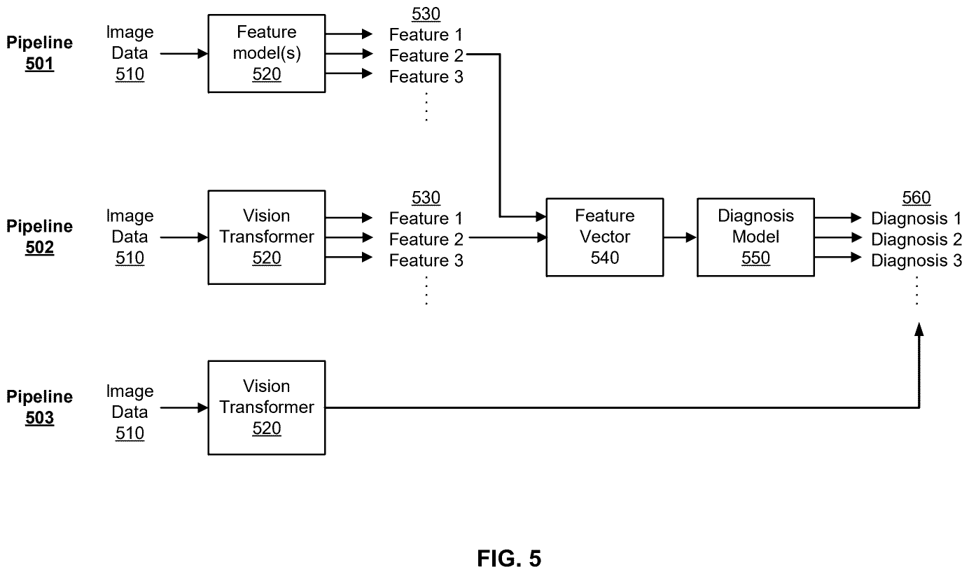

The pipeline determination module 230 receives the image from the client device 110 or the image store 140 and determines which of a set of pipelines to apply to the image. A pipeline may include a series of machine learning or deep learning models that the disease diagnosis tool 130 uses to autonomously determine a diagnosis from the image. For example, a pipeline may include a vision transformer followed by a diagnosis model, a feature model followed by a diagnosis model, or any other combination of one or more models. The pipeline may also include processing steps, such as dividing images into tiles, computing embeddings of images, or any other processing steps described in this disclosure. Various pipelines are described in further detail with respect to FIG. 5 below. Moreover, pipeline determination module 230 is optional, in that a pipeline may be hardwired or predetermined and therefore there may be no need to determine which pipeline to use in various implementations.

In some embodiments, the pipeline determination module 230 selects a pipeline to apply to the image based on a resolution of the image. The pipeline determination module 230 determines that the image has a high resolution based on the image having a resolution equal to or above a threshold and determines that the image has a low resolution based on the image having a resolution below the threshold. For high-resolution images, the pipeline determination module 230 may select a pipeline that includes a vision transformer. A vision transformer (ViT) is an image classification model that employs a transformer-based architecture. Much like how a transformer breaks text into a series of tokens, a vision transformer breaks an image into a series of smaller segments called “patches.” Vision transformers are particularly adept at image classification but are also computationally expensive to train and deploy. The vision transformer's attention layers, for example, compute interactions between every pair of patches. The pipeline determination module 230 may determine to process low-resolution images with vision transformers as well or, to reduce the computational expense of processing multiple images, the pipeline determination module 230 may determine that low-resolution images may be processed with a feature model. A feature model may be model that is less computationally demanding, for example a convolutional neural network (CNN). Vision transformers and feature models are described in further detail with respect to the classification module 234.

The image preprocessing module 231 adjusts the image before it is further processed. In some embodiments, the image preprocessing module 231 adjusts the size of the image by cropping the image. For example, the image preprocessing module 231 may crop an image that is 3000×3000 pixels to a size of 2000×2000 pixels. The image preprocessing module 231 may crop the image to a region of interest. For example, for an image of a retina, the image preprocessing module 231 may crop the image such that the retina takes up the entire image and extra space surrounding the retina is cropped out. The image preprocessing module 231 may adjust the size of the image by upscaling the image. For example, the image preprocessing module 231 may upscale an image that is 1500×1500 pixels to 2000×2000 pixels. In some embodiments, the image preprocessing module 231 may adjust the size of the image such that is a standard size. For example, the image preprocessing module 231 may crop or upscale an image to be a standard size of 2000×2000 pixels. In some embodiments, the image preprocessing module 231 performs a combination of cropping and upscaling an image. For example, the image preprocessing module 231 may crop the image to a region of interest, determine whether the size of the region of interest is greater or less than the size of the standard size, and either further crop or upscale the image to meet the standard size.

The image tiling module 232 receives an image and divides the image into a set of tiles. A tile is a section of the image, where the dimensions of the section are smaller than the dimensions of the image as a whole. To use an example, the image tiling module 232 may split an image that has dimensions of 2000×2000 pixels into a 16×16 grid of tiles, where each of the 256 tiles has dimensions of 125×125 pixels. In some embodiments, the image is of a standard size. For example, the image may be adjusted by the image preprocessing module 231. In these embodiments, the image tiling module 232 may divide the image into a fixed number of tiles of a fixed dimension.

In some embodiments, the image tiling module 232 divides the image into tiles such that each tile partially overlaps with one or more adjacent tiles (e.g., tiles above, below, to the left, or to the right). For example, a first tile may overlap with a second tile of the same size such that the rightmost column of pixels in the first tile is identical to the leftmost column of pixels in the second tile. Overlaps may be determined by a stride length, the stride length indicating an amount to progress along an axis for a next tile. For example, a stride length of 0.5 may cause each tile to overlap with at least half of another tile.

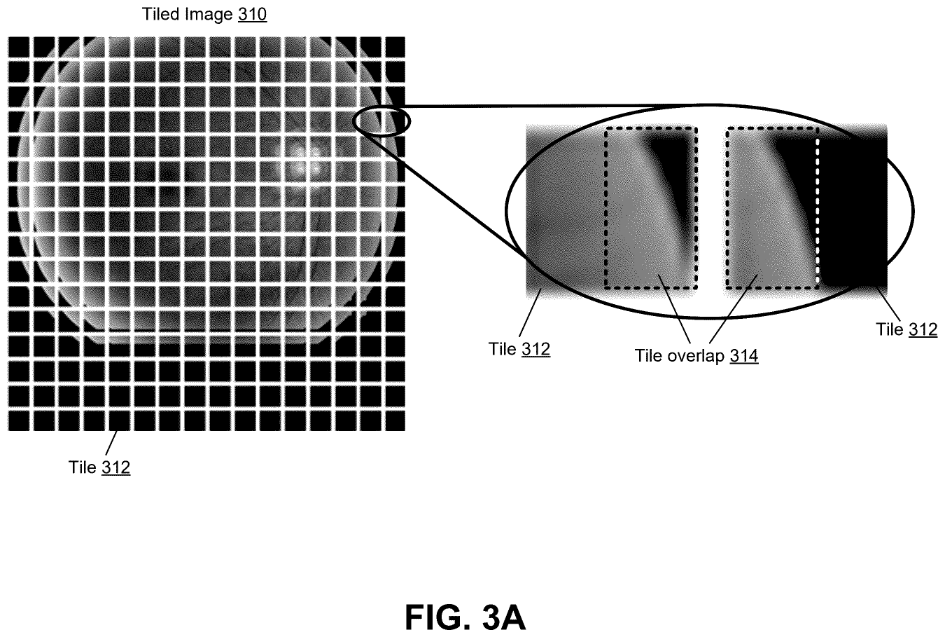

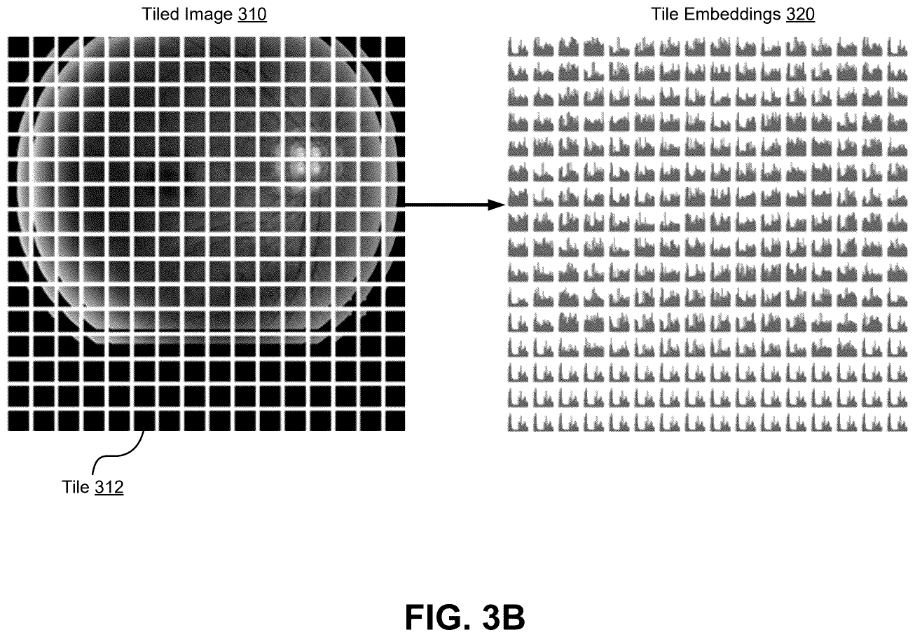

Turning briefly away from FIG. 2 to illustrate tiling, FIG. 3A illustrates an example tiled image for an image of a body part. The left side of FIG. 3A depicts a tiled image 310. The tiled image 310 is a high-resolution image of an eye that the image tiling module 232 has divided into a 16 by 16 grid of tiles. Each tile 312 has dimensions smaller than the dimensions of the tiled image 310. For example, the dimension of each tile 312 is 256×256 pixels. The right side of FIG. 3A depicts a close-up of two tiles 312 from the tiled image 310. The two tiles 312 are overlapped such that the right side of one tile 312 is identical to the left side of the other tile 312. The region where the two tiles 312 overlap is depicted by a dotted border and labeled as the tile overlap 314. The stride length of the two overlapping tiles 312 is 0.5, or 128 pixels.

In some embodiments, the tiles generated by the image tiling module 232 may be input directly into a vision transformer. As described earlier, the vision transformer's attention layers compute interactions between every pair of inputs. In this case, the vision transformer's attention layers would compute interactions between every pair of tiles. However, there are technological disadvantages to having the disease diagnosis tool process high-resolution images. If the image tiling module 232 divides high resolution images into small tile sizes, the number of tiles increases, requiring the vision transformer's attention layers to compute a large number of interactions. For example, if the image tiling module 232 divides a 2000×2000 pixel image into tiles of size 16×16 pixels, the number of tiles produced is 125×125, or 15,625 tiles. That means that the attention layers would need to make 15,6252² comparisons (around 2.5E8 comparisons), which is computationally expensive and inefficient. As an embodiment to address this issue, the image tiling module 232 may alternatively downsize high-resolution images and generate a smaller number of tiles. For example, a 2000×2000 pixel high-resolution image may be downsized to a 224×224 image and then tiled into 256 tiles of size 14×14 pixels. However, downsizing images presents yet another problem; it results in the loss of valuable image information in the high-resolution image, which reduces accuracy in diagnoses and therefore cannot practically be used where diagnosis accuracy is paramount for patient health.

The tile embedding module 233 reduces the size of a high-resolution image by generating an embedding for each tile of the high-resolution image. An embedding of a tile is a numerical representation of the tile in a N-dimensional space (e.g., an embedding space or latent space). For example, while a tile may be a two-dimensional array of pixel values, an embedding of the tile may be a one-dimensional vector. Tiles that are more similar will have vectors that are closer in the embedding space while tiles that are less similar will have vectors that are farther in the embedding space. While an embedding of a tile has lower dimensions than the tile, it is not a down-sampled version of the tile. The embedding of the tile compresses the important feature information of the tile, particularly feature information that makes the tile similar or different to other tiles in the image. As such, generating an embedding of a tile of a high-resolution image does not lose important feature information the way downsizing the tile would.

The tile embedding module 233 generates a tile embedding for each tile using an embedding model. The embedding model receives the tiles as input and produces, as an output, a representation of each tile as a N-dimensional vector in an embedding space. An example of an embedding model could be a convolution neural network (CNN) or a vision transformer. FIG. 3B illustrates example tile embeddings for an image of a body part. Each of the tile embeddings 320 corresponds to a tile 312 of the tiled image 310.

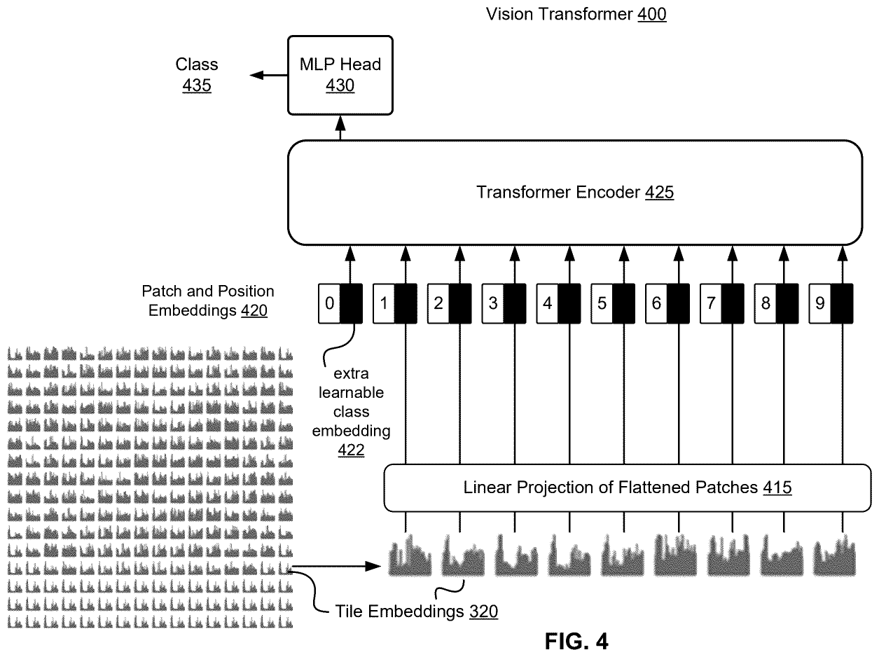

The classification module 234 determines a diagnosis of a disease based on the image of the body part. In some embodiments, the classification module 234 determines the diagnosis using a vision transformer. A vision transformer (ViT) is an image classification model that employs a transformer-based architecture. Much like how a transformer breaks text into a series of tokens, a vision transformer breaks an image into a series of smaller segments called “patches.” The vision transformer model may be stored in model store 241. FIG. 4 illustrates an example vision transformer. The classification module 234 provides the vision transformer 400 with an input. In some embodiments, such as when the image has low resolution, the vision transformer 400 receives a set of patches as input. In other embodiments, such as when the image has high resolution, the vision transformer 400 receives a set of tile embeddings 320 as input.

The vision transformer 400 passes the tile embeddings 320 through a linear projection layer 415, producing patch embeddings. A patch embedding for a tile represents the image content of the tile. That is, tiles with similar image content will have similar patch embeddings. For example, as overlapping tiles have similar image content, they are likely to have similar patch embeddings. The vision transformer 400 generates position embeddings for each tile. A position embedding for a tile represents a location of the tile in the high-resolution image. Tiles that are closer together in the image will have similar position embeddings, regardless of whether the tiles display similar image content. The vision transformer 400 pre-appends an extra learnable class embedding 422 to the position embeddings.

The vision transformer 400 may generate a position embedding of a tile using an embedding model that receives the tile as input and produces, as an output, a representation of the tile as a vector in the embedding space. The patch embeddings and position embeddings are in the same embedding space and, as such, have the same dimensions. The vision transformer 400 sums the patch and positional embeddings to generate the patch and position embeddings 420. The transformer encoder 425 of the vision transformer 400 is made up of a series of transformer blocks. Each transformer block includes attention layers and a multilayer perceptron (MLP) component, which includes layers that are used for classification. The transformer encoder 425 receives the patch and position embeddings 420 as input. The output of the transformer encoder 425 is passed through an MLP head 430. The MLP head 430 outputs a class 435 as output. The class 435 may be a set of features identified in the image (e.g., biomarkers) or a diagnosis of a disease.

In some embodiments, the vision transformer is trained to identify features of the image. A feature refers to an object within an image. Objects may include anatomical objects, such as blood vessels, organs, or optic nerves. Objects may also include biomarkers, abnormalities relative to a normal anatomic part of a human being. Example biomarkers are lesions, fissures, and dark spots. A feature vector refers to a data structure that includes one or more different features. The feature vector may map the different features to auxiliary information. For example, where the image data includes images corresponding to different locations of a body part, the feature vector may map the features identified from those images to their respective different locations of the body part. As an example, where the images are retinal images, and one image is taken for each quadrant of a retina, the feature vector may include four data points, the data points including respective features identified in an image of each of the four quadrants. The classification module 234 inputs the tile embeddings generated by the tile embedding module 233 into the transformer layer of the vision transformer and receives, as output from the vision transformer, features in the image and their positions. For example, the vision transformer 400 of FIG. 4 may output class 435 that includes identified features and their positions in the image. As another example, the classification module 234 may input tile embeddings associated with an image of an eye and receive an output indicating positions of optic nerves. Training data for the vision transformer may be stored in training data 240.

In embodiments where the vision transformer is trained to identify features of the image, the classification module 234 performs the task of determining a diagnosis. The classification module 234 determines the diagnosis by inputting features into a diagnosis model. The features may be a subset of the features identified by the vision transformer, for example biomarkers and their locations. A diagnosis model is a model trained to, based on an input feature vector, output a prediction of a disease indicated by the image. For example, the diagnosis model may receive a feature vector including optic nerves identified in an image of an eye and output a prediction that the image indicates glaucoma. The diagnosis model may be any machine learning model (e.g., deep learning model, convolutional neural network (CNN), etc.). The training data may include data manually labeled by doctors or others trained to diagnose diseases. A manual label of a disease may be paired with image feature vectors, optionally with other patient data. Image labels may also indicate various stages of diseases, or where in the image bio-markers are located. In some embodiments, the diagnosis model may output probabilities corresponding to predicted diseases or may output predicted diseases that have probabilities exceeding a threshold. The diagnosis model may be stored in the model store 241. Training data for the diagnosis model may be stored in training data 240. Further discussion of how a diagnosis model reaches a diagnosis from features is disclosed in commonly-owned U.S. Pat. No. 12,051,490, filed Dec. 3, 2021, issued Jul. 30, 2024, the disclosure of which is hereby incorporated by reference herein in its entirety.

In some embodiments, the vision transformer is trained to determine a diagnosis of a disease based on the image. That is, rather than using the vision transformer to identify features of the image and using a diagnosis model to predict a disease indicated by the image, the classification module 234 uses the vision transformer to perform the task of determining a diagnosis of a disease. That is, the class output of the vision transformer is a diagnosis of a disease rather than a set of features (e.g., biomarkers) identified in the image. In such embodiments, the classification module 234 inputs the tile embeddings generated by the tile embedding module 233 into the transformer layer of the vision transformer and receives, as output from the vision transformer, a prediction of a disease indicated in the image. For example, the classification module 234 may input tile embeddings associated with an image of an eye and receive an output indicating the disease of glaucoma. Training data for the vision transformer may be stored in training data 240.

In some embodiments, the classification module 234 determines the diagnosis using a feature model. A feature model is a machine learning model trained to identify or extract one or more features in an image. A feature model may be any machine learning model such as a deep learning model, a convolutional neural network (CNN), an ensemble model, and so on. The feature model may be trained using labeled training images, where the training images show at least portions of human anatomy, and are labeled with at least a score (e.g., likelihood or probability) of whether the image includes a biomarker (e.g., feature). In an embodiment, the labels may include an identification of one or more specific biomarkers within the image. The labels may include additional information, such as other objects within the images and one or more body parts that the training image depicts. Further discussion of the structure, training, and use of feature models is disclosed in commonly-owned U.S. Pat. No. 10,115,194, filed Apr. 6, 2016, issued Oct. 30, 2018, the disclosure of which is hereby incorporated by reference herein in its entirety.

In some embodiments, the classification module 234 selects a feature model to apply to the image from a set of feature models. For example, the classification module 234 may determine a body part that corresponds to the image and select a feature model trained on images and diseases specific to a body part. For example, the feature model may identify that an image corresponds to an eye and may apply a feature model trained be trained to identify eye diseases from images of eyes. The classification module 234 may select a feature model to apply to an image on bases other than a body part depicted in the image, such as on any other characteristics of the patient (e.g., a specific age range). The classification module 234 may apply a feature model that is fine-tuned using data of a specific patient. Feature models may be stored in model store 241. Training data for feature models may be stored in training data 240.

The classification module 234 may provide the feature model with full images or image tiles as input and receive, as output, data representative of one or more corresponding features. This data may include probabilities that the image data includes one or more features or may include a binary determination that certain feature(s) are included in the image data. The classification module 234 determines the diagnosis by inputting the features identified by the feature model into a diagnosis model.

In some embodiments, rather than using a two-stage model (e.g., the feature model followed by the diagnosis model), a single stage model may be used to directly predict a diagnosis for a patient from the image data. Any form of machine learning model trained to output a diagnosis directly based on image data may be used to determine one or more diagnoses.

FIG. 5 illustrates example pipelines for determining a diagnosis from an image of a body part. The pipelines, discussed in detail in the preceding description of the classification module 234, include various combinations of vision transformers, feature models, and diagnosis models. Pipelining allows for the disease diagnosis tool 130 to optimize use of computational resources. The digital diagnostic tool can select which of a set of pipelines to use to best process an image, depending on the resolution of the image, or the computational demands of the various models.

The first pipeline, pipeline 501, illustrates an embodiment where the classification module 234 processes an image using a feature model and a diagnosis model. The classification module 234 inputs image data 510 into a feature model 520, which outputs a set of features 530. The classification module 234 forms a feature vector 540 from the set of features 530 and provides the feature vector 540 to a diagnosis model 550. The diagnosis model 550 receives the feature vector 540 as input and outputs one or more diagnoses 560 autonomously.

The second pipeline, pipeline 502, illustrates an embodiment where the classification module 234 processes an image using a vision transformer trained to output features and a diagnosis model. The classification module 234 inputs image data 510 into the vision transformer 520. In this pipeline, the vision transformer 520 is trained to receive tiles (e.g., image tiles or embeddings of image tiles) as input and output a set of features 530 identified in the image. The classification module 234 forms a feature vector 540 from the set of features 530 and provides the feature vector 540 to a diagnosis model 550. The diagnosis model 550 receives the feature vector 540 as input and outputs one or more diagnoses 560 autonomously.

The third pipeline, pipeline 503, illustrates an embodiment where the classification module 234 processes an image using a vision transformer trained to directly output diagnoses. The classification module 234 inputs image data 510 into the vision transformer 520. In this pipeline, the vision transformer 520 is trained to receive tiles (e.g., image tiles or embeddings of image tiles) as input and output one or more diagnoses 560 autonomously.

The disease diagnosis tool 130 may select pipeline 501 to process images that are of low-resolution and select either of pipelines 502 or pipeline 503 to process high-resolution images. Vision transformers, which are excluded from pipeline 501, are computationally expensive to deploy. While it may be less computationally expensive to use a vision transformer to process a low-resolution image than a high-resolution image, low resolution images may be more easily and more cheaply processed by machine learning models that are not transformer models. The feature model and diagnosis model, for example, may be convolutional neural networks (CNN). In selecting a machine learning model like a CNN to process some images and using a vision transformer to process other images, the disease diagnosis tool 130 reduces the overall computational expense involved in processing multiple images.

The disease diagnosis tool 130 may select either of pipelines 502 or 503 depending on what the vision transformer is trained to output—features or a diagnosis. Pipeline 502 provides an advantage over pipeline 503 in that a resulting diagnosis is more explainable. That is, medical experts may validate that biomarkers output by the vision transformer would likely be indicators of a diagnosis produced by the diagnosis model. Pipeline 503 may be less computationally demanding than pipeline 502, as only one model is used rather than two, however it may be more difficult to verify that the vision transformer is identifying relevant features involved in making a diagnosis.

The heatmap module 235 generates a heatmap that shows where in the image the vision transformer placed attention. The heatmap may be used by medical experts to confirm the accuracy of the vision transformer in making an autonomous diagnosis. Namely, the heatmap allows medical experts to verify that the areas of the image where the vision transformer placed attention are areas relevant to making an autonomous diagnosis. Medical experts may identify areas of high attention that are irrelevant to making an autonomous diagnosis and provide feedback to the disease diagnosis tool 130 (e.g., through the client device 110). This allows the disease diagnosis tool 130 to retrain the vision transformer to be more accurate in identifying relevant features and to eliminate any possibility of bias in an autonomous diagnosis. Bias in an autonomous diagnosis may include instances where characteristics of a patient that are irrelevant to the disease are used in making the diagnosis. Such characteristics may include skin color, gender information, and so on. For example, if the vision transformer places high attention on an area of an image that includes skin tone, the heat map will display the area of high attention, providing an opportunity for a medical expert to identify the area as irrelevant to making an autonomous diagnosis.

To generate a heatmap, the heatmap module 235 extracts, for each tile of the image, one or more weights that the attention layers of the transformer applied to the tile. The heatmap module 235 generates an attention score for the tile based on the one or more weights that the attention layers applied. For example, if a first attention layer applied a weight of 0.2 and a second attention layer applied a weight of 0.4, the heatmap module 235 may generate an attention score for the tile that is the average of the two weights, 0.3, the sum of the two weights, 0.6, or any other combination of the weights. For instance, the heatmap module 235 may more heavily consider weights applied by later attention layers. The heatmap module 235 generates the heatmap for the image as a two-dimensional image that has pixels corresponding to the tiles of the input image, with each tile having an amplitude based on the attention score for the tile. The heatmap module 235 may display the heatmap on a display of the client device 110 (e.g., on a graphical user interface).

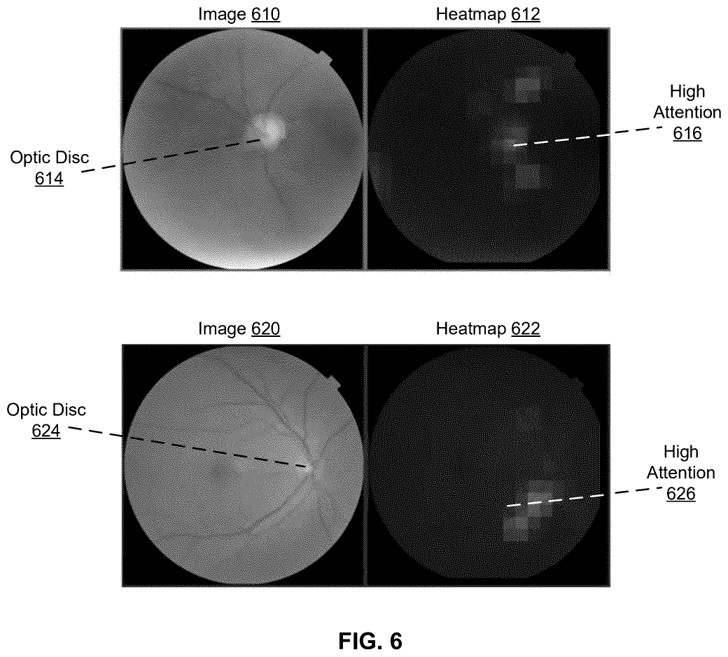

FIG. 6 illustrates example heatmaps for images of eyes. Images 610 and 620 respectively correspond to heatmaps 612 and 622. The pixels of the heatmaps 612 and 622 are shaded corresponding to the amplitudes of the attention scores for corresponding pixels in images 610 and 620. In this example, lighter pixels indicate that the vision transformer placed more attention in that area of the image. The top image 610 illustrates an eye with a damaged optic disc 614. The damage is due to glaucoma, a common optic disc disorder. The corresponding heatmap 612 includes a region of lighter pixels that are located around the optic disc 614. This indicates that the vision transformer placed high attention 616 in the region of the optic disc 614. If the vision transformer were to output a diagnosis of glaucoma for the image, a doctor would be able to verify that the vision transformer placed attention in the appropriate positions for identifying glaucoma (e.g., around the optic disc). The bottom image 620 illustrates with an optic disc 624 that is not damaged. The eye in image 620 does not show signs of glaucoma. The heatmap 622 indicates that the vision transformer did not place much attention around the optic disc, but instead placed high attention 626 on other bio-markers.

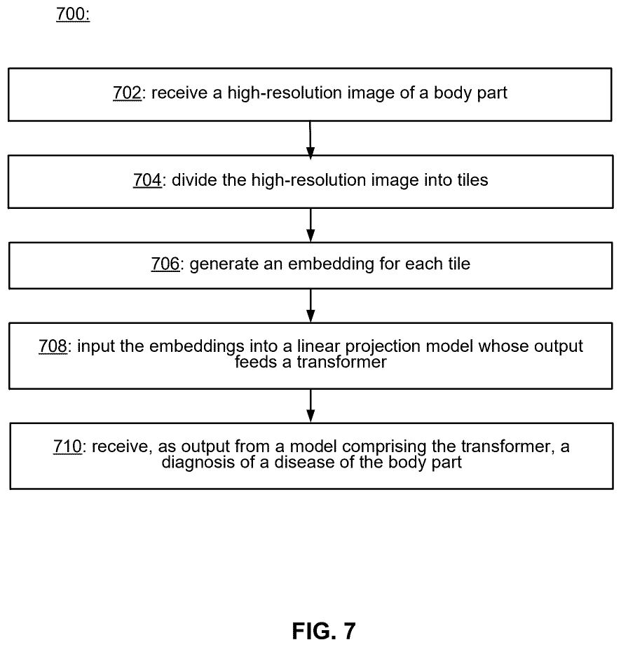

FIG. 7 is a flowchart of an exemplary process for using a vision transformer to produce a diagnosis from high-resolution images, in accordance with an embodiment. Process 700 begins with the disease diagnosis tool 130 receiving 702 a high-resolution image of a body part (e.g., using pipeline determination module 230). The disease diagnosis tool 130 divides 704 the high-resolution image into a plurality of tiles (e.g., using the image tiling module 232). The disease diagnosis tool 130 generates 706 a plurality of embeddings comprising an embedding for each tile of the plurality of tiles (e.g., using the tile embedding module 233). Each embedding has a position encoding corresponding to its tile's position in the image. The disease diagnosis tool 130 inputs 708 the plurality of embeddings into a linear projection model whose output feeds a transformer (e.g., using the classification module 234). The disease diagnosis tool 130 receives, as output from a model comprising the transformer, a diagnosis of a disease of the body part (e.g., using the classification module 234).

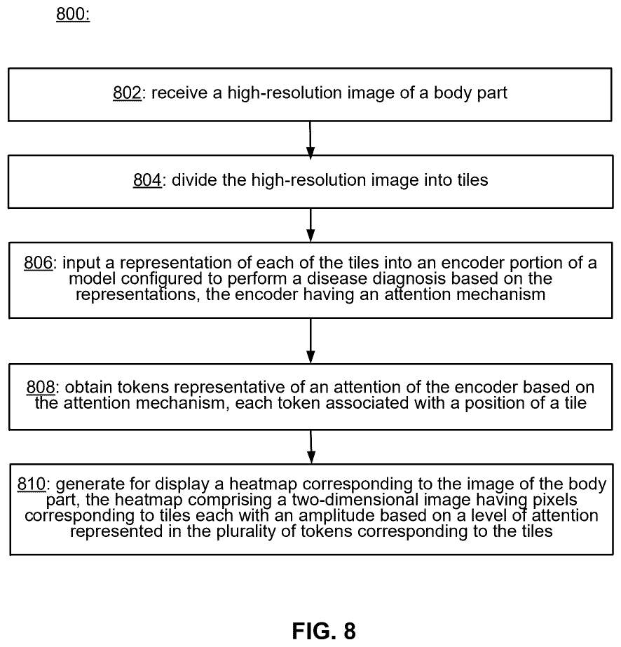

FIG. 8 is a flowchart of an exemplary process for generating a heatmap representative of attention placed on pixels of an image by a vision transformer, in accordance with an embodiment. Process 800 begins with the disease diagnosis tool 130 receiving 802 a high-resolution image of a body part (e.g., using pipeline determination module 230). The disease diagnosis tool 130 divides 804 the high-resolution image into a plurality of tiles (e.g., using the image tiling module 232). The disease diagnosis tool 130 inputs 806 a representation of each of the plurality of tiles into an encoder portion of a model (e.g., using the tile embedding module 233 and the classification module 234). The model is configured to perform a disease diagnosis based on the representations. The encoder has an attention mechanism. The disease diagnosis tool 130 obtains 808 a plurality of tokens representative of an attention of the encoder based on the attention mechanism (e.g., using the heat map module 235). Each token is associated with a position of a tile of the plurality of tiles. The image classification module 130 generates 810 for display a heat map corresponding to the image of the body part (e.g., using the hat map module 235). The heat map includes a two-dimensional image having pixels corresponding to tiles. Each pixel has an amplitude based on a level of attention represented in the plurality of tokens corresponding to the tiles.

The above disclosure describes examples pertaining to diagnosing diseases from eye images, however the digital diagnostic tool 130 may be used to diagnosis any type of disease or medical condition from an image of any type of the body. For example, the digital diagnostic tool 130 may receive an image of skin and determine a diagnosis of acne, psoriasis, eczema, rosacea, etc. As another example, the digital diagnostic tool 130 may receive an x-ray image of a bone and identify a fracture. Additionally, the systems and methods of the above disclosure may be applied in non-medical contexts. For example, the process of inputting tile embeddings rather than the tiles themselves into a vision transformer may be applied to any high-resolution image, not just high-resolution images of body parts.

The foregoing description of the embodiments of the invention has been presented for the purpose of illustration; it is not intended to be exhaustive or to limit the invention to the precise forms disclosed. Persons skilled in the relevant art can appreciate that many modifications and variations are possible in light of the above disclosure.

Some portions of this description describe the embodiments of the invention in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. In one embodiment, a software module is implemented with a computer program product comprising a computer-readable medium containing computer program code, which can be executed by a computer processor for performing any or all of the steps, operations, or processes described.

Embodiments of the invention may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Embodiments of the invention may also relate to a product that is produced by a computing process described herein. Such a product may comprise information resulting from a computing process, where the information is stored on a non-transitory, tangible computer readable storage medium and may include any embodiment of a computer program product or other data combination described herein.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention, which is set forth in the following claims.

What is claimed is:

1. A method for validating an autonomous diagnosis of a patient, the method comprising:
receiving a high-resolution image of a body part;
dividing the high-resolution image into a plurality of tiles;
inputting a representation of each of the plurality of tiles into an encoder portion of a model, the model configured to perform a disease diagnosis based on the representations, and the encoder having an attention mechanism;
obtaining a plurality of tokens representative of an attention of the encoder based on the attention mechanism, each token associated with a position of a tile of the plurality of tiles; and
generating for display a heat map corresponding to the image of the body part, the heat map comprising a two-dimensional image having pixels corresponding to tiles each with an amplitude based on a level of attention represented in the plurality of tokens corresponding to the tiles.

2. The method of claim 1, further comprising:
determining that the high-resolution image is high-resolution based on it having a resolution above a threshold resolution, wherein images having a resolution below the threshold resolution are used to perform diagnosis based on extracting features from the images using a feature extraction model and inputting the extracted features into a diagnostic model.

3. The method of claim 1, wherein each tile is at least partially overlapping with at least one other tile.

4. The method of claim 1, wherein each tile is overlapping with at least half of at least one other tile.

5. The method of claim 1, wherein the pixels of the heat map are arranged to reflect a collection of tiles that contributed to the diagnosis of the disease of the body part.

6. A non-transitory computer-readable medium comprising memory with instructions encoded thereon for validating an autonomous diagnosis of a patient, the instructions when executed causing one or more processors to perform operations, the instructions comprising instructions to:
receive a high-resolution image of a body part;
divide the high-resolution image into a plurality of tiles;
input a representation of each of the plurality of tiles into an encoder portion of a model, the model configured to perform a disease diagnosis based on the representations, and the encoder having an attention mechanism;
obtain a plurality of tokens representative of an attention of the encoder based on the attention mechanism, each token associated with a position of a tile of the plurality of tiles; and
generate for display a heat map corresponding to the image of the body part, the heat map comprising a two-dimensional image having pixels corresponding to tiles each with an amplitude based on a level of attention represented in the plurality of tokens corresponding to the tiles.

7. The non-transitory computer-readable medium of claim 6, the instructions further comprising instructions to:
determining that the high-resolution image is high-resolution based on it having a resolution above a threshold resolution, wherein images having a resolution below the threshold resolution are used to perform diagnosis based on extracting features from the images using a feature extraction model and inputting the extracted features into a diagnostic model.

8. The non-transitory computer-readable medium of claim 6, wherein each tile is at least partially overlapping with at least one other tile.

9. The non-transitory computer-readable medium of claim 6, wherein each tile is overlapping with at least half of at least one other tile.

10. The non-transitory computer-readable medium of claim 6, wherein the pixels of the heat map are arranged to reflect a collection of tiles that contributed to the diagnosis of the disease of the body part.

11. A method comprising:
receiving a high-resolution image;
dividing the high-resolution image into a plurality of tiles;
inputting a representation of each of the plurality of tiles into an encoder portion of a model, the model having an attention mechanism;
obtaining a plurality of tokens representative of an attention of the model based on the attention mechanism, each token associated with a position of a tile of the plurality of tiles; and
generating for display a heat map, the heat map comprising a two-dimensional image having pixels corresponding to tiles each with an amplitude based on a level of attention represented in the plurality of tokens corresponding to the tiles.

12. The method of claim 11, further comprising:
determining that the high-resolution image is high-resolution based on it having a resolution above a threshold resolution, wherein images having a resolution below the threshold resolution are used to perform a task based on extracting features from the images using a feature extraction model and inputting the extracted features into a task-specific model.

13. The method of claim 11, wherein each tile is at least partially overlapping with at least one other tile.

14. The method of claim 1, wherein each tile is overlapping with at least half of at least one other tile.

15. The method of claim 1, wherein the pixels of the heat map are arranged to reflect a collection of tiles that contributed to classification by the model.

16. A non-transitory computer-readable medium comprising memory with instructions encoded thereon that, when executed, cause one or more processors to perform operations, the instructions comprising instructions to:
receive a high-resolution image;
divide the high-resolution image into a plurality of tiles;
input a representation of each of the plurality of tiles into an encoder portion of a model, the model having an attention mechanism;
obtain a plurality of tokens representative of an attention of the model based on the attention mechanism, each token associated with a position of a tile of the plurality of tiles; and
generate for display a heat map, the heat map comprising a two-dimensional image having pixels corresponding to tiles each with an amplitude based on a level of attention represented in the plurality of tokens corresponding to the tiles.

17. The non-transitory computer-readable medium of claim 16, the instructions further comprising instructions to:
determine that the high-resolution image is high-resolution based on it having a resolution above a threshold resolution, wherein images having a resolution below the threshold resolution are used to perform a task based on extracting features from the images using a feature extraction model and inputting the extracted features into a task-specific model.

18. The non-transitory computer-readable medium of claim 16, wherein each tile is at least partially overlapping with at least one other tile.

19. The non-transitory computer-readable medium of claim 16, wherein each tile is overlapping with at least half of at least one other tile.

20. The non-transitory computer-readable medium of claim 16, wherein the pixels of the heat map are arranged to reflect a collection of tiles that contributed to classification by the model.
